(19) United States
(12) Patent Application Publication
(10) Pub. No.: US 2007/0078442 A1
(43) Pub. Date: Apr. 5, 2007

(54) TAPERED ATTACHMENT FOR PLEURAL CATHETER

(76) Inventor: Martin L. Mayse, University City, MO (US)

Correspondence Address: CHARLES C. MCCLOSKEY, 763 S. NEW BALLAS ROAD STE. 170, ST. LOUIS, MO, 63141, US

(21) Appl. No.: 11/466,604

(22) Filed: Aug. 23, 2006

Related U.S. Application Data
(60) Provisional application No. 60/710,480, filed on Aug. 24, 2005.

(51) Int. Cl. A61M 39/00

(52) U.S. Cl. 604/533

(57) ABSTRACT

The present invention is a tapered attachment for use with a pleural catheter that connects securely to the pleural catheter and has a hollow elongated member and a tapered distal portion. When the tapered attachment connected to the prior art catheter, the hollow elongated member opens the self-sealing valve of the prior art catheter allowing communication from the interior of the pleural catheter to the interior of the tapered attachment. The distal outer taper of the invention can connect to a variety of standard drainage systems to remove fluids or gases from the pleural cavity. The invention may also have a one way valve. With the invention in place and functioning, air does not enter the pleural space. Additionally, the variations in pleural pressure that normally occur during respiration can pump fluids or gases out of the pleural cavity through the invention.

### CROSS REFERENCE TO RELATED APPLICATION

This non-provisional application claims priority to the provisional application for patent Ser. No. 60/710,480 which was filed on Aug. 24, 2005 which is incorporated by reference and the aforesaid application is commonly owned by the same inventor.

### BACKGROUND OF THE INVENTION

The present invention generally relates to medical devices utilized in removal of fluid or gases from the pleural or peritoneal cavity of a person. More specifically, the present invention relates to a tapered attachment for use with the prior art catheter described in U.S. Pat. No. 5,484,401, not owned by this inventor. The tapered attachment connects securely to the prior art catheter and opens the self-sealing valve of a prior art catheter. The distal outer taper of the tapered attachment can be connected to a variety of drainage systems to aid in the removal of fluid or gases from the pleural space. Alternatively, the tapered attachment can also have a one-way valve which allows passage of fluid or gases out of the pleural cavity while preventing entry of air into the pleural cavity.

The prior art catheter, of U.S. Pat. No. 5,484,401, implants in the pleural space for extended periods of time and intermittently drains pleural effusion fluids. This prior art catheter has an elongated flexible tube with openings, or fenestrations, along the proximal portion and a self-sealing valve on the distal end. The fenestrations receive fluids and gases from the pleural space, or cavity. The self-sealing valve end provides an automatic closure of the flow path from the pleural cavity through the catheter and when so closed prevents drainage of fluid from the pleural cavity and introduction of air into the pleural cavity. A drainage flow path from the pleural cavity begins by insertion of an elongated hollow tub into the self-sealing valve of the prior art pleural catheter, thus opening the self sealing valve. With the self-sealing valve opened by such an elongated hollow tube, negative pressure can be applied to the flow path to remove fluid or gases from the pleural cavity.

While the prior art catheter is effective, it exhibits drawbacks. For example, the prior art catheter has a design for dedicated drainage systems. Connecting the prior art catheter to standard drainage systems in various medical settings is quite cumbersome and causes some risk to patient safety. Also, if the self-sealing valve of the prior art catheter opens and negative pressure is absent or not maintained on the flow path, air will enter the pleural cavity and increase the risk of lung collapse. The present invention overcomes these limitations of the prior art catheter and improves both the functionality and the safety of connecting to catheters.

### SUMMARY OF THE INVENTION

The tapered attachment of the present invention connects securely to the prior art catheter, includes a hollow elongated member which opens the self-sealing valve, and has a tapered distal portion which can connect to standard drainage systems. When the tapered attachment connects to a prior art catheter, the hollow elongated member of the tapered attachment opens the self-sealing valve of the prior art catheter for communication of fluids and gases from the pleural catheter through the tapered attachment. The distal end of the tapered attachment then connects to a variety of standard drainage systems to remove fluids and gases from the pleural cavity as directed by a medical provider such as a physician.

A one-way valve can also be provided within the tapered attachment as an alternate embodiment. The one-way valve allows passage of fluids and gases out of the pleural cavity but not the entry of air into the pleural cavity. During usage, when the tapered attachment is in place on a patient and is inadvertently opened to the atmosphere, gases and fluids may drain from the pleural cavity, but air does not enter the pleural space thus maintaining lung function. Additionally, the variations in pleural pressure that normally occur during respiration, or breathing, can pump fluids and gases out of the pleural cavity.

It is, therefore, the principal object of this invention to provide an attachment for a catheter that readily secures upon the catheter and communicates to standard drainage systems.

Another object of this invention is to provide an attachment for a catheter that opens valves contained with the catheter.

A further object of this invention is to provide an attachment for a catheter that does not admit air through the attachment and into the pleural cavity.

These and other objects may become more apparent to those skilled in the art upon review of the summary of the invention as provided herein. In addition, the invention will be better understood upon undertaking a study of the description of its preferred embodiment, in view of the drawings.

### BRIEF DESCRIPTION OF THE DRAWINGS

In referring to the drawings,

FIG. 1 is an elevation view of the prior art catheter described in U.S. Pat. No. 5,484,401;

FIG. 2 is a cross-section view of the self-sealing valve of the prior art catheter;

FIG. 3 is a longitudinal cross-section view of the present invention;

FIG. 4 is a side view of the present invention;

FIG. 5 is an exploded view of a tapered attachment of the present invention;

FIG. 6 is a partial cross-section view of the tapered attachment of FIG. 3, connected to the prior art catheter of FIG. 1 opening the self-sealing valve, shown as 72, of FIG. 2; FIGS. 7a, 7b are longitudinal cross-section views of the tapered attachment showing alternate embodiments of the present invention including a “duckbill” type one-way valve in FIG. 7A and a disc type one-way valve in FIG. 7B; and,

FIG. 8 is an elevated view of the tapered attachment with a luer fitting allowing for disassembly into two parts.

The same reference numerals refer to the same parts throughout the various figures.

### DESCRIPTION OF THE PREFERRED EMBODIMENTS

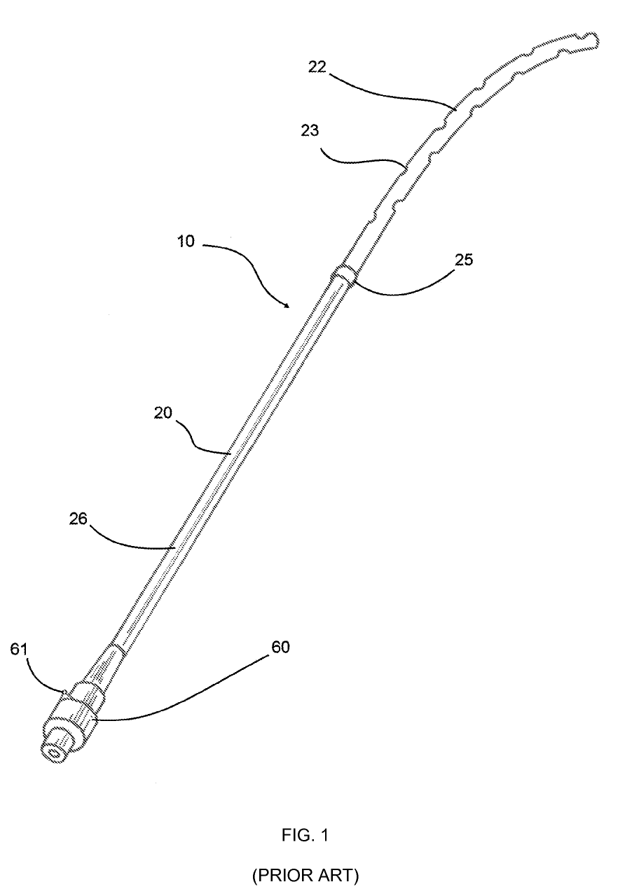

In reference to the drawings, FIG. 1 shows an elevational view of the prior art catheter 10 described in U.S. Pat. No. 5,484,401. The prior art catheter 10 has an elongated catheter 20 with a proximal end 22 and a distal end 26. The proximal end is fenestrated, or made to have pores, with a series of holes 23 or openings allowing communication between the environment outside, or exterior, of the catheter and the lumen, or internal bore of a catheter. A cuff 25 is located near the mid-portion of the elongated catheter 20, distal to the holes 23 in the proximal end 22 of the elongated catheter 20 and is fixedly attached to the elongated catheter 20. A self-sealing valve 60 is fixedly attached to the distal end 26 of the catheter 20. The prior art catheter is implanted into the pleural space of a patient by a physician using procedures known in the art, with the proximal portion 22 in the pleural cavity, the cuff 25 within the chest wall, and the distal end 26 and the self-sealing valve 60 located outside the body.

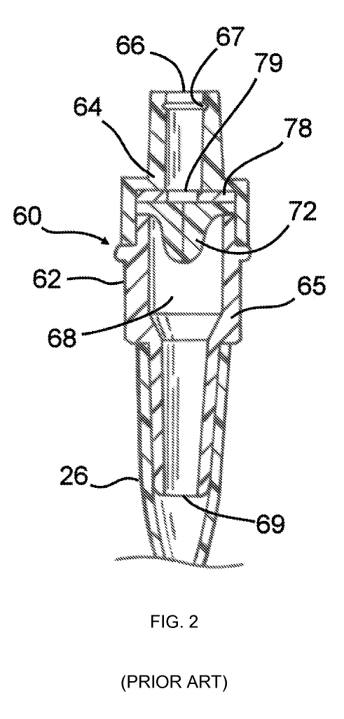

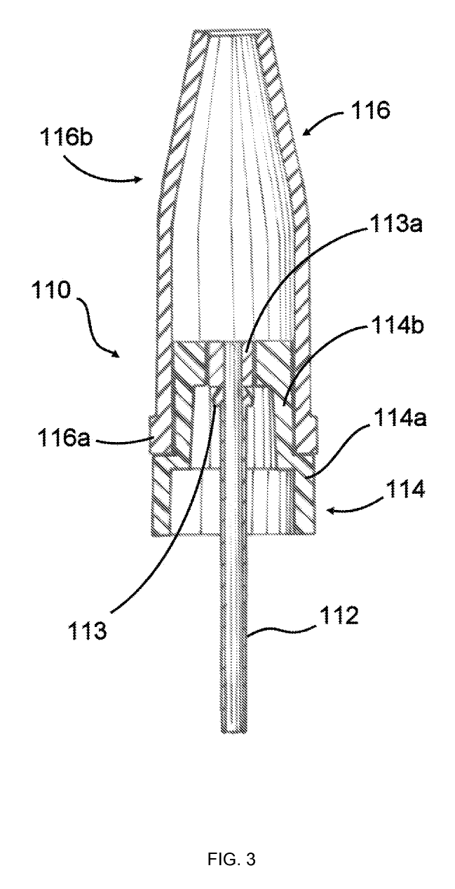

FIG. 2 shows a cross-sectional view of a prior art self-sealing valve 60 made in accordance with the principles of U.S. Pat. No. 5,484,401. The valve includes a valve body 62 having a distal portion 64 and a proximal portion 65 fixedly attached to one another. The proximal portion 65 of the self-sealing valve 60 inserts into the distal portion 26 of the elongated catheter 20 and is fixedly attached to the elongated catheter 20. The end 66 of the distal portion 64 of the valve body 62 and the end 69 of the proximal portion 65 of the valve body 62 each have a hole, and the centers of the portions 64, 65 are hollowed out, thereby forming a passage 68 through the valve body 62. Positioned within this passage 68 is a “duckbill” valve 72 which is of the type known in the art consisting of an elastomeric, molded, one-piece dome containing a slit in the center of the dome. The duckbill valve 72 may be opened by inserting an elongated member through the passageway 68 from the end 66 of the distal portion 64 of the valve body 62 to pry apart the duckbill valve 72. Adjacent to the duckbill valve 72 toward the distal portion 64 of the valve body 62 is an elastomeric seal 78. The elastomeric seal 78 is a disk-shaped element having a hole 79 through the center to seal against the outside of the hollow elongated member 112 of the tapered attachment 110 as shown in FIG. 3.

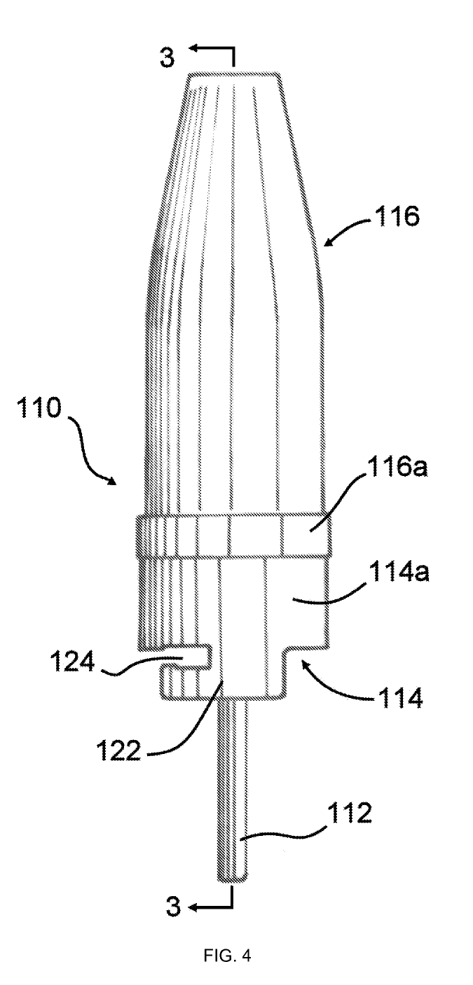

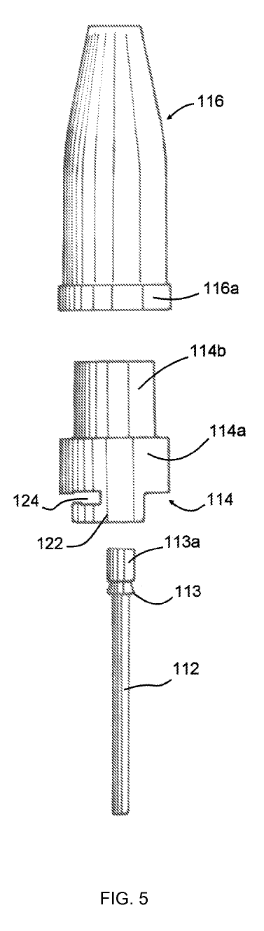

FIG. 3 shows a longitudinal cross sectional view, FIG. 4 shows a side view, and FIG. 5 shows an exploded view of the tapered attachment 110 of the current invention that has a hollow elongated member 112, a hollow locking portion 114, and a hollow tapered portion 116. When assembled as shown in these figures, the hollow elongated member 112 fixedly attaches to the hollow locking portion 114 which in turn fixedly attaches to the hollow tapered portion 116 of the tapered attachment 110. The hollow elongated member is generally a tube of thin, but strong wall construction. The hollow elongated member 112 generally defines the longitudinal axis of the present invention and extends to approximately the center of the invention. Towards one end of the hollow elongated member 112 a ridge 113 extends radially outwards upon the exterior of the member. The ridge has a somewhat triangular shape in cross section and a certain diameter to ease fitting with the self sealing valve 60 of the catheter 10 as later described.

Outside of the hollow elongated member 112, the present invention has the locking body 114. The locking body 114 has a generally cylindrical form coaxial with the elongated member 112. The locking body 114 has a lower portion 114a that is a hollow cylinder with an outer diameter similar to that of the tapered portion 116 and an inner diameter similar to that of the distal portion of the self sealing valve 60. As later shown in FIG. 4, the lower portion 114a has a means to temporarily secure the present invention upon the prior art catheter. The lower portion 114a is generally coaxial with the elongated member 112 which passes through the lower portion 114a spaced away from the walls of the hollow cylinder. Upon the lower portion 114a, the locking body has the upper portion 114b. The upper portion 114b is generally cylindrical in shape and has an outer diameter less than that of the lower portion 114a. The outer diameter of the upper portion 114b fits snugly within the tapered portion 116 connecting to it. The upper portion 114b is also coaxial with the elongated member 112 and has an end of the elongated member 112 opening therethrough into the interior of the tapered portion 116. The tapered portion 116 locating upon the upper portion 114b of the locking body 114, has a generally hollow cylindrical form with a ridge 114a upon one end and a tapering, as at 116b, of the cylindrical form generally towards the opposite end. The taper reaches its narrowest diameter at slightly less than the diameter of a tube of a standard drainage system. The ridge 116a meets the tapered member 116 at the step in diameter of the locking portion 114 where the lower portion 114a joins to the upper portion 114b.

With the elongated member 112 denoting the central axis of the present invention, FIG. 4 shows the invention assembled. The tapered portion 116 connects to the locking portion 114 where the ridge 114a abuts the lower portion 114a. The lower portion 114a has a lock beneath a portion of its circumference. The lock has an extension 122 that extends along an arc below the bottom edge of the lower portion 114a. The extension 122 is spaced apart from the lower portion 114a and forms a notch 124. The notch 124 has an arc like shape that corresponds with the bottom edge of the lower portion 114a. The notch 124 receives a partial flange 61 presently on the self-sealing valve 60 of the prior art catheter 10.

FIG. 5 shows the three components of the present invention. The tapered portion 116 has its exterior ridge 116a that connects to the locking portion 114 at the change in diameter between the lower portion 114a and the upper portion 114b. The diameter change is shown here in FIG. 5. As previously described, the elongated member 112 has a ridge 113 upon one end. In this figure, the ridge 113 appears as the lower portion of a sleeve 113a placed upon the exterior of the elongated member at one end. The sleeve locates the ridge away from the opening of the end so the ridge engages the interior of the self-sealing valve 60.

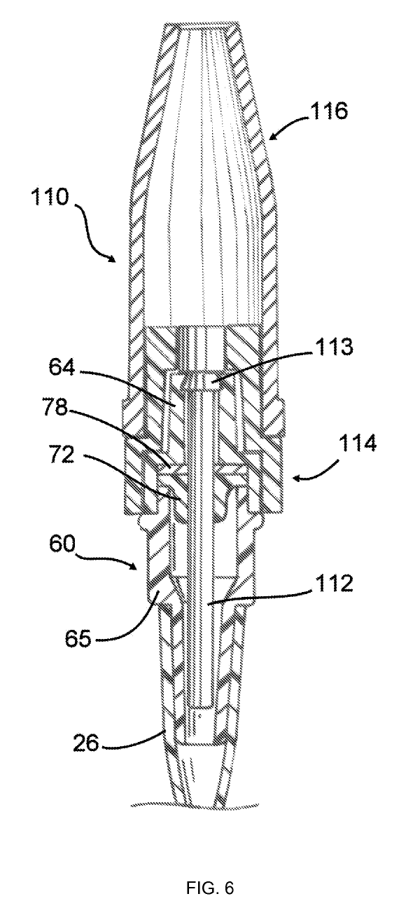

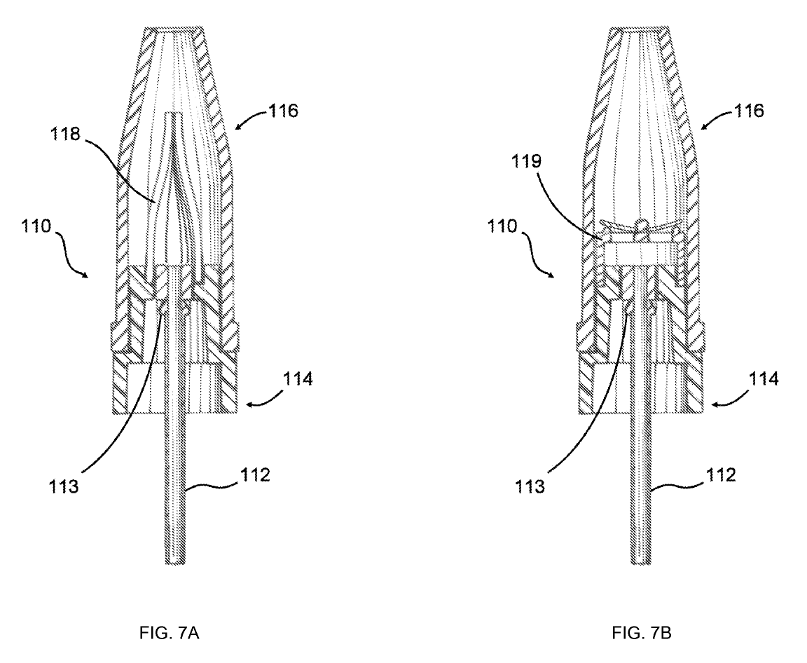

To use the tapered attachment 110 with the prior art catheter 10, the hollow elongated member 112 of the tapered attachment 110 is inserted into the hollow end 66 of the proximal portion 64 of the body 62 of the self-sealing valve 60 as shown in cross section in FIG. 6. When the tapered attachment 110 is inserted fully into the self-sealing valve 60, the ridge 113 of the hollow elongated member 112 engages the matched recess 67 in the proximal portion 64 of the body 62 of the self-sealing valve 60. The tapered attachment 110 can then be rotated such that the notch 124 in the extension 122 of the locking portion 114 engages the partial flange 61 in the proximal portion 64 of the body 62 of the self-sealing valve 60, thereby reversibly locking the tapered attachment 110 to the valve 60 of the prior art catheter 10. The hollow elongated member 112 of the tapered attachment 110 is slightly larger in its outside diameter than the hole 79 in the elastomeric seal 78 of the self-sealing valve 60, thereby ensuring that a seal is created between the elastomeric seal 78 and the outside of the hollow elongated member 112 of the tapered attachment 110 to prevent fluids and gases from leaking. The insertion of the hollow elongated member 112 of the tapered attachment 110 into the hollow end 66 of the proximal portion 64 of the body 62 of the self-sealing valve 60 opens the duckbill valve 72 and thereby allows access to the interior of the elongated catheter 20. The hollow tapered portion 116 of the tapered attachment 110 then connects to the tubing of a standard drainage system to aid in the removal of fluid or air from the pleural space.

The fluid removal procedure is discontinued by rotating the tapered attachment 110 such that the notch 124 in the extension 122 of the locking portion 114 of the tapered attachment 110 disengages the partial flange 61 on the proximal portion 64 of the body 62 of the self-sealing valve 60, thereby unlocking the tapered attachment 110 from the valve 60 of the prior art catheter 10 and then simply withdrawing the hollow elongated member 112 of the tapered attachment 110 from the self-sealing valve 60. As the end of the hollow elongated member 112 of the tapered attachment 110 exits the duckbill valve 72, the valve closes and prevents further fluid and gases from flowing out of the self-sealing valve 60 and also prevents air from entering the elongated catheter 20 and thereby the pleural cavity.

Alternatively, the tapered attachment 110 could also have other one-way valves as shown in FIGS. 7A, 7B. FIG. 7A shows the tapered attachment 110 modified to include a “duckbill” type one-way valve 118, similar to those known in the art, located within the hollow tapered portion 116 of the tapered attachment 110 and secured to the intern end of member 112. FIG. 7B shows the tapered attachment 110 modified to include an elastomeric disc type one-way valve 119, similar to those known in the art, located within the hollow tapered portion 116 of the tapered attachment 110. These one-way valves 118 and 119 are oriented such that flow is allowed to occur only in the direction from the hollow elongated member 112 toward and through the hollow tapered portion 116. If the tubing of the drainage system becomes inadvertently disconnected from the tapered portion 116 of the tapered attachment 1 10, the one-way valves 118 and 119 prevent air from entering the pleural space. Additionally, the one-way valves 118 and 119 allow the variations in pleural pressure that occur with normal breathing to pump fluid or gases out of the pleural cavity. It should be noted that a variety of other one-way valve designs could be substituted for the “duckbill” one-way valve 118 in FIG. 7A or the elastomeric disc type one-way valve 119 in FIG. 7B without deviating from the intent of this disclosure.

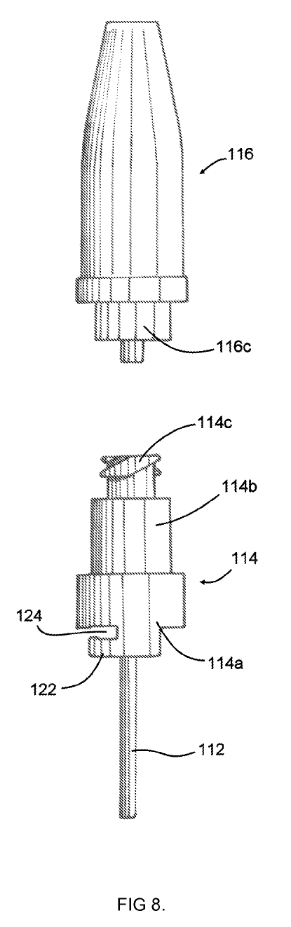

FIG. 8 shows an elevated view of the tapered attachment 110 where the upper portion 114b of the locking portion 114 has a female luer type connector with external locking threads 114c that are mated to internal locking threads (not shown) on a matched male luer type connector 116c on the distal end of the hollow tapered portion 116. This configuration allows removal of the hollow tapered portion 116 the locking portion 114 and permits other devices with a male luer type connector to attach to the locking portion 114 of the tapered attachment 110.

Variations or modifications of the subject matter of this invention may occur to those skilled in the art upon reviewing the disclosure provided herein. Such variations or modifications are intended to be encompassed within the scope of the invention as described herein. The description of the preferred embodiment and of the drawings showing the same are provided herein for illustrative purposes only.

From the aforementioned description, a tapered attachment for a pleural catheter has been described. The tapered attachment is uniquely capable of readily connecting a prior art catheter to a standard drainage system found in a medical setting. The tapered attachment and its various components may be manufactured from many materials including but not limited to polymers, high density polyethylene HDPE, polypropylene PP, polyethylene terephalate ethylene PETE, polyvinyl chloride PVC, nylon, ferrous and non-ferrous metals, their alloys and composites.

The phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. A connector that joins a catheter to a drainage system, said connector comprising:
a locking portion adapting to connect to the catheter, said locking portion having a lower portion and an integral opposite upper portion;
an elongated member, being hollow, coaxial with said locking portion and securing to said upper portion, said elongated member opening through said upper portion; and,
a tapered portion, generally flexible and hollow, overlapping said upper portion, and connecting to the drainage system, thus permitting communication from the catheter through said elongated member, said locking portion, and through said tapered portion.

2. The catheter connector of claim 1 further comprising:
said lower portion being a hollow cylinder and having a thin wall in proportion to the diameter of said lower portion; and,
said upper portion being a hollow cylinder and having a wall of lesser diameter than said lower portion, said upper portion and said lower portion forming a step proximate said lower portion.

3. The catheter connector of claim 2 wherein said lower portion has a means to secure said locking portion upon the catheter.

4. The catheter connector of claim 3 further comprising:
said securing means having a lock upon said lower portion, said lock engaging the catheter.

5. The catheter connector of claim 2 further comprising:
said elongated member having a sleeve upon one end, said sleeve having a ridge positioned towards the interior of said elongated member;
said upper portion having an opening centered therethrough; and,
said sleeve fitting snugly within said opening thereby aligning said elongated member axially with said locking portion.

6. The catheter connector of claim 4 further comprising:
said tapered portion having a leur lock engaging said upper portion of said locking member.

7. The catheter connector of claim 4 wherein said lock is a leur lock.

8. The catheter connector of claim 5 further comprising:
a valve communicating with said elongated member and said tapered portion, and said valve connecting to said sleeve.

9. The catheter connector of claim 8 wherein said valve is a one way valve.

10. The catheter connector of claim 9 wherein said valve is a one of a duck bill valve or a disc valve.
